(19) United States
(12) Patent Application Publication
(10) Pub. No.: US 2026/0273012 A1
(43) Pub. Date: Sep. 17, 2026

(54) Camellia Oleifera Tea Oil Concentrate and Uses Thereof

(71) Applicant: Laboratoires Expanscience, Paris La Defense Cedex (FR)

(72) Inventors: Sophie LECLERE, Dreux (FR); Gaetan BOYER, Coulombs (FR); Gaelle BELLEMERE, Champhol (FR); Stephanie BREDIF, Croisilles (FR)

(21) Appl. No.: 19/147,836

(22) PCT Filed: Feb. 16, 2024
(86) PCT No.: PCT/EP2024/054043

§ 371 Date: Jul. 14, 2025

(30) Foreign Application Priority Data
Feb. 16, 2023 (FR) FR2301466

(51) Int. Cl. A61K 36/82; A61K 8/9789; A61K 9/00; A61P 17/04; A61Q 19/00; A61Q 19/08; C11B 3/00; C11B 3/12

(52) U.S. Cl. CPC A61K 36/82; A61K 8/9789; A61K 9/0014; A61P 17/04; A61Q 19/005; A61Q 19/007; A61Q 19/08; C11B 3/006; C11B 3/12; A61K 2236/10; A61K 2236/35

(57) ABSTRACT

The invention relates to a lipid extract of Camellia oleifera tea oil concentrated in its unsaponifiable fraction, to a cosmetic, pharmaceutical or dermatological composition comprising a suitable excipient and said lipid extract, and to the uses thereof.

### TECHNICAL FIELD OF THE INVENTION

The invention relates to a lipid extract of Camellia oleifera tea oil, concentrated in its unsaponifiable fraction, to a cosmetic, nutraceutical, pharmaceutical or dermatological composition comprising a suitable excipient and said lipid extract.

Also an object of the invention is a method for preparing a lipid extract of Camellia oleifera tea oil, concentrated in its unsaponifiable fraction. The invention also relates to such a composition or such an extract for its use in the prevention or treatment of disorders or pathologies of the skin, mucous membranes or appendages. The invention finally relates to a cosmetic care method for the skin, appendages or mucous membranes, with a view to improving their condition or appearance, consisting of administering such a composition or such an extract, in particular in order to improve the physical/physiological and psychological well-being of the user.

### STATE OF THE ART

The genus Camellia is native to eastern Asia and comprises over 200 evergreen woody species. Some species have significant economic value, such as C. sinensis, C. japonica, and C. oleifera. C. sinensis is grown primarily in tropical and subtropical regions for tea, C. japonica is grown in temperate regions as an ornamental, and its oil is conventionally used as a cosmetic in Japan. C. oleifera, also known as oil-tea camellia, can grow on barren land without fertilizer, begin producing fruit six years after initial planting, and remain highly productive for 80 years. This species is mainly cultivated in China for the production of edible oils (“camellia oil,” “tea seed oil”) (Yang et al., 2016).

The amount of oil contained in seeds and the fatty acid composition can vary depending on the variety, soil and climate (Hu & Yang, 2018). Wen et al. (2018) estimate that light intensity during fruit development and ripening (July-October) influences fruit yield and quality.

Regarding varietal diversity, Yang et al. (2016) compared 10 different cultivars and seeds from several wild individuals and concluded that the oil content and fatty acid profiles of their extracts were not influenced by artificial cultivation and cultivar selection.

Camellia oleifera oil is used in cooking. It is also conventionally used to treat stomach aches and burns. Because of its monounsaturated fatty acid content (C18:1 predominantly) similar to olive oil, several studies have been conducted to determine whether it could be attributed the same health benefits, particularly in reducing cardiovascular risks. Studies have demonstrated the ability of tea oil to reduce cholesterol, triglycerides, and low-density lipoprotein levels, as well as thromboxane B2 and lipid peroxidation products. It also increases the activity of superoxide dismutase (SOD) and glutathione peroxidase (GSH-Px). Some of these activities may be due to the presence of saponins.

The Applicant has discovered that the lipid extract of Camellia oleifera tea oil, concentrated in its unsaponifiable fraction, has cosmetic and dermatological properties never described until now.

In particular, this is the first time that such lipid extracts have been used as such, for their specific properties. The Applicant has thus surprisingly discovered that the lipid extract of Camellia oleifera tea oil, concentrated in its unsaponifiable fraction, can be used to stimulate, restore or regulate the metabolism of skin and mucous membrane cells and/or for its use in the prevention and/or treatment of disorders related to skin tissue.

### SUMMARY OF THE INVENTION

According to a first aspect, the invention relates to a lipid extract of tea oil, said lipid extract being a Camellia oleifera tea oil, concentrated in its unsaponifiable fraction, containing from 1.5% to 100% by weight of unsaponifiables, preferably 1.8% to 80%, preferentially 2% to 60%, relative to the total weight of the extract.

According to a second aspect, the invention relates to a method for preparing said extract comprising the following successive steps:
a) Molecular distillation of a crude or refined oil of Camellia oleifera tea;
b) where applicable, extraction of the unsaponifiable matter;
c) recovery of the concentrated unsaponifiable oil obtained following step a) or of the unsaponifiable matter obtained following step b);
d) optionally a step of deodorization and/or decolorization of the concentrated unsaponifiable oil or the unsaponifiable matter.

Advantageously, step b) comprises the following successive steps:
i. saponification of concentrated tea oil into its unsaponifiable fraction obtained following step a),
ii. then extraction of the unsaponifiable matter using a suitable solvent, such as ethyl acetate and hexane.

According to a third aspect, the invention relates to a composition comprising, as active ingredient, said lipid extract of tea oil and a suitable excipient. The composition may be cosmetic, nutraceutical, pharmaceutical or dermatological.

According to a fourth aspect, the invention relates to the extract according to the invention or a composition comprising the extract according to the invention or the extract capable of being obtained by the method according to the invention, for its use in a dermatological or pharmaceutical composition.

According to a fifth aspect, the invention relates to the use of the extract according to the invention or of a composition comprising the extract according to the invention or the extract capable of being obtained by the method according to the invention, in a nutraceutical composition or as a food supplement.

According to a sixth aspect, the invention relates to a method for cosmetic care of the skin and/or appendages and/or mucous membranes with a view to improving their condition and/or their appearance, comprising the topical or oral administration of an extract according to the invention or of a composition according to the invention.

According to a seventh aspect, the invention relates to a method for the cosmetic treatment of dry skin, xerosis, sensitive skin, irritated skin, comprising the administration, topically or orally, of an extract according to the invention or of a composition according to the invention.

### DETAILED DESCRIPTION OF THE INVENTION

The object of the invention is a lipid extract of tea oil, said lipid extract being a Camellia oleifera tea oil concentrated in its unsaponifiable fraction, containing from 1.5% to 100% by weight of unsaponifiables, preferably 1.8% to 80%, preferentially 2% to 60%, relative to the total weight of the extract.

Unsaponifiable matter is the fraction of a fatty substance which, after prolonged action of an alkaline base, remains insoluble in water and can be extracted by an organic solvent. Five major groups of substances are present in most unsaponifiable matter of vegetable oils: saturated or unsaturated hydrocarbons, aliphatic or terpenic alcohols, sterols, tocopherols, tocotrienols, carotenoid pigments and xanthophylls.

The oil is concentrated in its unsaponifiable fraction, in particular it comprises more than 1.5% by weight of unsaponifiables, relative to the total weight of the extract, advantageously more than 2%, preferably more than 2.5% by weight of unsaponifiables.

The lipid extract of Camellia oleifera tea oil, concentrated in its unsaponifiable fraction, advantageously comprises fatty acids having from 12 to 22, more advantageously from 16 to 20, carbon atoms. These fatty acids can be saturated, monounsaturated or polyunsaturated. This concentrate is advantageously obtained from a refined tea oil. The oil can be refined according to methods known to the person skilled in the art such as physical refining (degumming with water, deacidification by deodorization by steam stripping) and chemical refining (degumming with water or acid treatment in order to remove phospholipids, neutralization of free fatty acids using a basic solution, decolorization, refrigeration and deodorization), in particular by deodorization by steam stripping. An example of characteristics of the refined Camellia oleifera tea oil is given in the following table.

TABLE 1
Fat cut (% by weight relative to the
total weight of the lipid extract)

| | |
|---|---|
| C16 (palmitic acid) | 5.0-10.0 |
| C16′ (5-hexadecenoic acid) | ≤1.0 |
| C18 (stearic acid) | ≤5.0 |
| C18′ (oleic acid) | 65-85 |
| C18″ (linoleic acid) | 6-12 |
| C18″′ (α-linolenic acid) | <1.0 |
| C20 (arachidic acid) | ≤1.0 |
| C20′ (eicasenoic acid) | ≤1.0 |
| C22 (behenic acid) | ≤1.0 |
| Tocopherol content (g/100 g) | 0.001-0.015 |
| Sterol content (g/100 g) | 0.1-0.6 |
| Squalene content (g/100 g) | 0.01-0.02 |
| Total content of unsaponifiables (g/100 g) | 0.4-0.8 |

From this oil, Camellia oleifera tea oil is advantageously concentrated into its unsaponifiable fraction—the “concentrate”, typically by molecular distillation. The oil concentrated into its unsaponifiable fraction advantageously has the following specifications.

TABLE 2
Fat cut (% by weight relative to the
total weight of the lipid extract)

| | |
|---|---|
| C16 (palmitic acid) | 5.0-10.0 |
| C16′ (5-hexadecenoic acid) | ≤1.0 |
| C18 (stearic acid) | ≤5.0 |
| C18′ (oleic acid) | 65-85 |
| C18″ (linoleic acid) | 6-12 |
| C18″′ (α-linolenic acid) | <1.0 |
| C20 (arachidic acid) | ≤1.0 |
| C20′ (eicasenoic acid) | ≤1.0 |
| C22 (behenic acid) | ≤1.0 |
| Total content of unsaponifiables (g/100 g) | 1.5-5 |

Advantageously, the unsaponifiable matter fraction is as described below.

TABLE 3
Unsaponifiable fraction (% by weight relative
to the total weight of the lipid extract)

| | |
|---|---|
| α-Tocopherol content g/100 g | 0.01-0.150 |
| Sterol content g/100 g | 1.0-4.5 |
| Relative % campesterol | 1-3.5% |
| Relative % stigmasterol | 1-3.0% |
| Relative % beta-sitosterol | 1-20% |
| Relative % ergosterol family | 1-2.5% |
| Relative % lanosterol | 2-10% |
| Relative % (beta- amyrin + delta5-avenasterol) | 20-35% |
| Relative % (lupeol + delta7-stigmasterol) | 20-35% |
| Squalene | 0.05-0.2 |
| Total unsaponifiable g/100 g | 1.5-5 |

Relative %: relative to the total weight of sterols

According to a preferred embodiment of the invention, the α-tocopherol content is greater than 60% by weight, relative to the total weight of tocopherols, preferably 80% by weight, preferentially 95% by weight.

According to another preferred embodiment, the sterol content of the extract according to the invention is greater than 1.5% by weight, preferably 2%, relative to the total weight of the extract.

The lipid extract according to the invention advantageously contains β-amyrin and 45-avenasterol, preferably from 10 to 50% by weight, preferentially from 15 to 40%, more preferentially from 15 to 35% by weight, typically from 20 to 35% by weight, relative to the total weight of sterols and/or lupeol and 47-stigmasterol, preferably from 10 to 50% by weight, preferentially from 15 to 40%, more preferentially from 15 to 35% by weight, typically from 20 to 35% by weight, relative to the total weight of sterols.

Advantageously, the lipid extract according to the invention comprises:

TABLE 4
% by weight relative to the total weight of sterols

| | |
|---|---|
| beta-amyrin + | 10-50% |
| delta5-avenasterol | preferably 15-40%, preferably 15-35% |
| lupeol + | 10-50% |
| delta7-stigmasterol | preferably 15-40%, preferably 15-35% |
| lanosterol | 2-20% |
| | preferably 3-15%, preferably 4-10% |
| beta-sitosterol | 5-30% |
| | preferably 10-25%, preferably 12-20% |

The extract according to the invention is advantageously obtained by a method comprising the following successive steps:
a) molecular distillation of a crude or refined oil of Camellia oleifera tea;
b) where applicable, extraction of the unsaponifiable matter;
c) recovery of the concentrated unsaponifiable oil obtained following step a) or of the unsaponifiable matter obtained following step b); and
d) optionally a step of deodorization and/or decolorization of the concentrated unsaponifiable oil or the unsaponifiable matter.

When the oil enriched (concentrated) in unsaponifiable matter is to be recovered, step b) is not carried out. When the unsaponifiable matter fraction is to be recovered, step b) is carried out.

This step b) advantageously comprises the following successive steps:
i. saponification of concentrated tea oil into its unsaponifiable fraction obtained following step a),
ii. then extraction of the unsaponifiable matter using a suitable solvent, such as ethyl acetate and hexane.

The invention also relates to a method for preparing an extract according to the invention comprising the following successive steps:
a) molecular distillation of a crude or refined oil of Camellia oleifera tea;
b) where applicable, extraction of the unsaponifiable matter;
c) recovery of the concentrated unsaponifiable oil obtained following step a) or of the unsaponifiable matter obtained following step b);
d) optionally a step of deodorization and/or decolorization of the concentrated unsaponifiable oil or the unsaponifiable matter.

The molecular distillation step a) is preferably carried out using a device selected from centrifugal type molecular distillers and scraped film type molecular devices.

Centrifugal molecular distillers are known to the person skilled in the art. For example, application EP 493 144 describes a molecular distiller of this type. Generally, the product to be distilled is spread in a thin layer on the heated surface (hot surface) of a conical rotor rotating at high speed. The distillation chamber is placed under vacuum. Under these conditions, there is evaporation and not boiling, from the hot surface, of the constituents of the oil such as the unsaponifiable matters, the advantage being that the oil and its constituents, in particular the unsaponifiable matters (these products being known to be fragile), are not degraded during evaporation.

Scraped film molecular distillers are also known to the person skilled in the art. Generally, they comprise a distillation chamber equipped with a rotating scraper, allowing the continuous spreading on the evaporation surface (hot surface) of the products to be distilled. The product vapors are condensed by means of a refrigerated finger, placed in the center of the distillation chamber. The peripheral feed and vacuum systems are very similar to those of a centrifugal distiller (feed pumps, vane and oil diffusion vacuum pumps, etc.). The recovery of residues and distillates is done by gravitational flow into suitable containers.

At the end of the fractioning step, the distilled fraction rich in unsaponifiables advantageously represents 3 to 15% by weight of the starting oil, and the distilled fraction rich in triglycerides advantageously represents 85 to 97% by weight of the starting oil.

It was further verified that this method did not cause any chemical modification or alteration of the unsaponifiable matter compounds, and that the highly unsaturated fractions were preserved. Therefore, the fatty acid distribution of concentrated tea oil is identical to that of tea oil before concentration.

Following this molecular distillation step, the product obtained may optionally be deodorized and/or decolorized by methods known to the person skilled in the art, for example deodorized by steam stripping or molecular distillation, or decolorized by treatment on activated carbon.

The tea oil concentrated in its unsaponifiable fraction thus obtained has the characteristics described above.

When it is desired to recover the oil enriched with unsaponifiable matter, step b) is not carried out.

When the unsaponifiable matter fraction is to be recovered, step b) is carried out. This step advantageously comprises the following successive steps:
i. saponification of concentrated tea oil into its unsaponifiable fraction obtained following step a),
ii. then extraction of the unsaponifiable matter using a suitable solvent, such as ethyl acetate and hexane.

The unsaponifiable matter of tea oil can be obtained by methods known to the person skilled in the art. For example, it can be obtained by carrying out saponification on tea oil concentrated in its unsaponifiable fraction, then by extracting this unsaponifiable matter using a suitable solvent. Typically, the unsaponifiable matter is then washed until the soaps are completely removed then the solvent is evaporated. Finally, the unsaponifiable matter advantageously undergoes deodorization with water vapor then stripping with nitrogen in order to eliminate traces of solvent.

The unsaponifiable matter of tea oil thus obtained advantageously has the characteristics described above.

In the remainder of the description, “extract according to the invention” will be considered to designate the extract as such, as defined above, or the extract capable of being obtained by the method according to the invention as described above.

Another object of the invention relates to a composition comprising, as active ingredient, a lipid extract of Camellia oleifera tea oil according to the invention.

The composition according to the invention advantageously comprises from 0.01% to 20%, advantageously from 0.05 to 15%, more advantageously 0.1 to 10%, by weight of said lipid extract, relative to the total weight of the composition.

The lipid extract according to the invention is advantageously used as an active agent in a composition such as a cosmetic, nutraceutical, dermatological or pharmaceutical composition, which may comprise one or more suitable excipients.

The composition according to the invention can be formulated in the form of different preparations suitable for topical or oral administration.

Alternatively, the various preparations are suitable for topical administration and include creams, emulsions, milks, ointments, lotions, oils, aqueous or hydro-alcoholic or glycolic solutions, powders, patches, sprays, shampoos, varnishes or any other product for external application.

In the context of cosmetic, pharmaceutical or dermatological use, the composition is advantageously formulated in the form of a preparation suitable for topical administration.

In the context of nutraceutical use, the composition is advantageously formulated in the form of a preparation suitable for oral administration. The extract according to the invention can be included either in a food supplement or in a nutraceutical composition. The food supplement can be in the form of the extract according to the invention as such or in the form of gelatin or vegetable capsules or soft capsules. Said food supplement can then contain from 10 to 100% by weight of the extract according to the invention.

The composition of the present invention can be incorporated directly and without further modification into nutraceuticals, dietary products, particularly high-protein ones, or beverages using techniques such as mixing, infusion, injection, blending, absorption, kneading, and spraying.

The composition according to the invention may also contain the usual adjuvants in the pharmaceutical, dermatological, cosmetic or nutraceutical fields, such as hydrophilic or lipophilic gelling agents, hydrophilic or lipophilic active ingredients, thickeners, preservatives, antioxidants, solvents, perfumes, chelating agents, odor absorbers, chemical or mineral filters, mineral pigments, surfactants, polymers, silicone oils and coloring matters. The quantities of these different adjuvants are those conventionally used in the fields considered, for example from 0.01% to 20% of the total weight of the composition. These adjuvants, depending on their nature, may be introduced into the fatty phase, into the aqueous phase, into the lipid vesicles and/or into the nanoparticles. The person skilled in the art is able to choose the appropriate adjuvants according to the intended application.

As oils which can be used in the compositions for implementing the invention, mention may be made of mineral oils, oils of vegetable origin (apricot oil, sunflower oil, plum oil), oils of animal origin, synthetic oils, silicone oils and fluorinated oils (perfluoropolyethers). Fatty alcohols (cetyl alcohol), fatty acids and waxes (beeswax) may also be used as fats.

As emulsifiers and co-emulsifiers which can be used in the invention, mention may be made, for example, of fatty acid and polyethylene glycol esters such as PEG-40 stearate, PEG-100 stearate, fatty acid and polyol esters such as glyceryl stearate and sorbitan tristearate.

As hydrophilic gelling agents, mention may be made in particular of carboxyvinyl polymers (carbomer), acrylic copolymers such as acrylate/alkylacrylate copolymers, polyacrylamides, polysaccharides, natural gums and clays, and, as lipophilic gelling agents, mention may be made of modified clays such as bentones, metal salts of fatty acids, hydrophobic silica and polyethylenes.

The administration modes, dosages and optimal galenic forms of the compositions according to the invention can be determined according to the criteria generally taken into account in establishing a pharmaceutical, dermatological or cosmetic treatment adapted to a patient, such as for example the extent of the area of skin to be treated, tolerance to the treatment, skin type.

The invention also relates to the use of an extract according to the invention or of a composition according to the invention as a food supplement or in a nutraceutical composition.

The invention also relates to an extract according to the invention or a composition according to the invention for its use as, or in, a dermatological or pharmaceutical composition.

The composition or extract according to the invention can be used to stimulate, restore or regulate the metabolism of skin and mucous membrane cells and/or in the prevention and/or treatment of disorders related to skin tissue. Advantageously, the composition or extract according to the present invention is used in the prevention and/or treatment of disorders or pathologies of the skin and/or mucous membranes and/or appendages.

Preferably, the disorders or pathologies of the skin and/or mucous membranes and/or appendages are selected from inflammatory reactions, disorders related to intrinsic or extrinsic stress such as psychological stress, stress related to a state of anxiety, stress related to radical attacks related or not to chemical or atmospheric pollution, and/or related to exposure to UV or IR, disorders of the barrier or homeostasis, photosensitized skin, mechanical and/or thermal aggressions, or as a healing agent. Disorders or pathologies of the skin and/or mucous membranes and/or appendages can also be selected from those caused by imbalances in the microbiota; they are advantageously selected from atopic dermatitis, eczema, the development of bad axillary odors, weakening of the skin barrier, acne, psoriasis, hidradenitis suppurativa, folliculitis, cradle cap, dandruff, itching, particularly of the scalp, irritations, candidiasis and bacterial vaginosis.

Advantageously, the inflammatory, irritative reactions, disorders or pathologies or disorders of the skin barrier or homeostasis are: acne, rosacea or erythrocouperose, vascular disorders, in particular redness and couperose, diaper dermatitis, atopic dermatitis, eczema, contact dermatitis, irritant dermatitis, allergic dermatitis, seborrheic dermatitis (cradle cap), sensitive skin, reactive skin, dry skin (xerosis), dehydrated skin, skin with redness, skin erythema, aged or photoaged skin, photosensitized skin, pigmented skin (melasma, post-inflammatory pigmentation, etc.), skin with stretch marks, sunburn, irritation by chemical or physical agents (for example stress for pregnant women), bacteriological, fungal, skin aging, in particular photoaging and disorders related to free radical attacks related to chemical or atmospheric pollution, and/or related to exposure to UV or IR.

Particularly advantageously, the disorders or pathologies of the skin and/or mucous membranes and/or appendages are selected from dry skin, xerosis, sensitive skin and irritations.

Advantageously, the invention also relates to an extract according to the invention or a composition according to the invention for preventing and/or treating dry skin, xerosis, sensitive skin, irritations, fragile lips, cheilitis, atopic dermatitis, eczema and psoriasis. These disorders may be skin disorders related to intrinsic or extrinsic stress such as psychological stress.

The composition or extract according to the invention advantageously allows to improve skin comfort.

According to a particular embodiment, the extract or composition according to the invention can be used as an anti-aging agent and/or healing agent; to prevent damage to the skin, and/or maintain the homeostasis of the skin or mucous membranes or as an antioxidant and/or anti-inflammatory agent. Advantageously, the composition or extract according to the invention can be used to prevent or delay premature skin aging, in particular photo-induced, advantageously to prevent, reduce and/or treat wrinkles, fine lines or an alteration of the microrelief.

The composition or extract according to the invention can also be used to promote healing. According to a particular technical characteristic, the composition or extract according to the invention can also be used in the prevention and/or treatment of pathologies or conditions selected from the group consisting of superficial scars, fragile lips and cheilitis, stretch marks, skin after bites, skin abrasions, spots and/or skin scabs, and fragile and sensitive skin, advantageously fragile and sensitive skin.

The invention also relates to the use of an extract according to the invention or of a composition according to the invention for the manufacture of a cosmetic or pharmaceutical or dermatological composition to prevent and/or treat disorders or pathologies of the skin and/or mucous membranes and/or appendages. Preferably, the disorders or pathologies of the skin and/or mucous membranes and/or appendages are selected from inflammatory reactions, disorders related to intrinsic or extrinsic stress such as psychological stress, stress related to a state of anxiety, stress related to radical attacks related or not to chemical or atmospheric pollution, and/or related to exposure to UV or IR, disorders of the barrier or homeostasis, photosensitized skin, mechanical and/or thermal aggressions.

The invention further relates to a method for preventing and/or treating disorders or pathologies of the skin and/or mucous membranes and/or appendages, comprising the administration, in particular the topical administration, of an effective amount of an extract according to the invention or of a composition according to the invention, to a subject in need thereof. Preferably, the disorders or pathologies of the skin and/or mucous membranes and/or appendages are as defined above.

Another object of the invention relates to a non-therapeutic cosmetic care method for the skin and/or appendages and/or mucous membranes with a view to improving their condition and/or their appearance, advantageously with a view to improving the firmness, elasticity or tone of the skin, comprising the topical or oral administration of an extract or a composition according to the invention.

Said cosmetic care method is preferably carried out with a view to improving skin comfort, typically on healthy skin, healthy appendages or healthy mucous membranes, particularly advantageously for treating and/or preventing dry skin, xerosis, sensitive skin and irritated skin.

The cosmetic uses and cosmetic care methods according to the present invention are typically carried out on healthy skin or healthy parts of the body and are not therapeutic.

Another object of the invention relates to a method for the cosmetic treatment of dry skin, xerosis, sensitive skin, irritated skin, comprising the administration, by topical or oral route, of an extract or a composition according to the invention.

Dry skin, xerosis, sensitive and irritated skin cause feelings of tautness, lack of comfort and suppleness of the skin.

### FIGURES

FIG. 1: Active ingredient vs placebo results for each of the ten parameters of the “sensitive scale” (left: active ingredient, right: placebo). Significance D0 vs D28: *=significant (α<0.05), ^{o}=borderline significant (0.05<α<0.1). Significance active ingredient vs placebo: solid line=significant (α<0.05), hatched line==borderline significant (0.05<α<0.1).

FIG. 2: Overall active ingredient vs placebo score of the “sensitive scale” (left: active ingredient, right: placebo). Significance D0 vs D28: *=significant (α<0.05), ^{o}=borderline significant (0.05<α<0.1). Significance active ingredient vs placebo: solid line=significant (α<0.05), hatched line==borderline significant (0.05<α<0.1).

FIG. 3: Evolution of vocal stress (left: active ingredient, right: placebo). Significance D0 vs D28: *=significant (α<0.05), ^{o}=borderline significant (0.05<α<0.1). Significance active ingredient vs placebo: solid line=significant (α<0.05), hatched line==borderline significant (0.05<α<0.1).

FIG. 4: Evolution of cardiac coherence (left: active ingredient, right: placebo). Significance DO vs D28: *=significant (α<0.05), ^{o}=borderline significant (0.05<α<0.1). Significance active ingredient vs placebo: solid line=significant (α<0.05), hatched line==borderline significant (0.05<α<0.1).

FIG. 5: Evolution of the electrodermal response (left: active ingredient, right: placebo). Significance D0 vs D28: *=significant (α<0.05), ^{o}=borderline significant (0.05<α<0.1). Significance active ingredient vs placebo: solid line=significant (α<0.05), hatched line==borderline significant (0.05<α<0.1).

FIG. 6: Evaluation of corneometry (left: active ingredient, right: placebo) and hydration (left: active ingredient, right: placebo). Significance D0 vs D28: *=significant (α<0.05), ^{o}=borderline significant (0.05<α<0.1). Significance active ingredient vs placebo: solid line=significant (α<0.05), hatched line==borderline significant (0.05<α<0.1).

### EXAMPLES

### Example 1: Concentrate According to the Invention

An example of a concentrate according to the invention is a refined oil of Camellia oleifera concentrated in its unsaponifiable fraction. The extract is prepared by molecular distillation.
Vacuum condition: 10⁻² to 10⁻³ mbar
Distillation body temperature between 25° and 280° C.

The mass distribution is shown in the table below.

TABLE 5
Fat cut (% by weight relative to the
total weight of the lipid extract)

| | |
|---|---|
| C16 (palmitic acid) | 11 |
| C16′ (5-hexadecenoic acid) | 0.1 |
| C18 (stearic acid) | 2.0 |
| C18′ (oleic acid) | 77 |
| C18″ (linoleic acid) | 9 |
| C18″′ (α-linolenic acid) | 0.2 |
| C20 (eicasenoic acid) | 0.1 |
| C20′ (eicasenoic acid) | 0.3 |
| C22 (behenic acid) | 0 |
| C22′ | 0 |
| Tocopherol content (g/100 g) | 0.07 |
| Sterol content (g/100 g) | 2.21 |
| Squalene content (g/100 g) | 0.07 |
| Total content of unsaponifiables (g/100 g) | 2.8 |

The mass distribution of tocopherols is shown in the table below.

TABLE 6

| | |
|---|---|
| % α-tocopherol | 100 |
| % β-tocopherol | 0 |
| % δ-tocopherol | 0 |
| % γ-tocopherol | 0 |

The mass distribution of sterols is shown in the table below.

TABLE 7

| | |
|---|---|
| % campesterol | 2.9 |
| % stigmasterol | 2.0 |
| % β-sitosterol | 14.0 |
| % ergosterol and derivatives | 2.0 |
| % lanosterol | 7.0 |
| Relative % (β-amyrin + Δ5-avenasterol) | 31.0 |
| Relative % (lupeol + Δ7-stigmasterol) | 29.5 |

### Example 2: In Vitro Biological Activity Tests of the Extract According to the Invention

The extract tested is the concentrate from Example 1. It will be referred to in the studies as “tea oil concentrate”. Unless otherwise indicated, the percentages are expressed as the weight of concentrate relative to the total weight of the composition tested.

### Anti-Inflammatory Activity

The anti-inflammatory activity of tea oil concentrate (THE) was investigated by assaying interleukin-1 alpha (IL-1 alpha) produced by keratinocytes in response to pro-inflammatory stress induced by PMA (Phorbol-Myristate-Acetate) in a normal human keratinocyte model, in a reconstructed epidermis model subjected to PMA stress, as well as in a reconstructed epidermis model subjected to SDS (Sodium Dodecyl Sulfate) stress.

### Effect in a Model of Normal Human Keratinocytes

### Materials and Methods

Normal human keratinocytes were pre-incubated for 24 hours in the presence of 0.001%, 0.005% or 0.01% tea tree oil concentrate or 0.1 μM dexamethasone, an anti-inflammatory reference. The cells were then stimulated by overnight treatment in the presence of 10 μg/mL PMA (Phorbol-Myristate-Acetate).

At the end of treatment, IL-1 alpha produced was quantified by ELISA assay in culture supernatants.

The results were statistically analyzed by one-way analysis of variance (ANOVA), followed by Tukey post-test.

### Results

Tea oil concentrate significantly inhibited PMA-induced IL-1 alpha production in keratinocytes; therefore, it has anti-inflammatory potential.

TABLE 8
Dosage of interleukin-1 alpha produced by keratinocytes
subjected to inflammatory stress induced by PMA

| | |
|---|---|
| IL-1 alpha | |
| (pg/mL) | Protection (%) |

| | | | |
|---|---|---|---|
| Control cells | 57.8 ± 9.8 | | |
| Stimulated cells (PMA | 92.1 ± 4.7 | | |
| 10 μg/ml) | | | |
| Reference | 66.6 ± 0.5 | 74% | p < 0.001 |
| (Dexamethasone 0.1 μM) | | | |
| THE 0.001% | 69.4 ± 4.4 | 66% | p < 0.01 |
| THE 0.005% | 45.3 ± 7.4 | 136% | p < 0.001 |

### Effect in a Reconstructed Epidermis Model Subjected to PMA Stress

### Materials and Methods

Reconstructed epidermis were treated topically with a formulation (cream) containing 1% tea tree oil concentrate or its placebo, or systemically with 1 μM dexamethasone, an anti-inflammatory reference. After 24 hours of pre-incubation, epidermis were stimulated with 0.5 μg/mL of PMA and incubated again for 24 hours.

Culture subnatants were collected then IL-1 alpha assay was performed.

The results were statistically analyzed using ANOVA followed by Tukey's test.

### Results

Tea oil concentrate, formulated at 1%, significantly inhibited PMA-induced IL-1 alpha release in reconstructed epidermis. This effect was significantly different from placebo.

TABLE 9
Dosage of IL-1 alpha produced by reconstructed
epidermis stimulated by PMA

| | Interleukin-1 | | | |
|---|---|---|---|---|
| | alpha | | | |
| | (pg/mL) | % Protection | | |
| Control cells | 122 ± 4 | | | |
| Stimulated cells | 533 ± 35 | | | |
| (PMA 0.5 μg/mL) | | | | |
| Reference | 334 ± 41 | 48% | p < 0.001 | |
| (Dexamethasone 1 μM) | | | | |
| Placebo | 246 ± 15 | 70% | p < 0.001 | p < 0.05 |
| THE 1% | 105 ± 11 | 104% | p < 0.001 | |

### Effect in a Reconstructed Epidermis Model Subjected to SDS Stress

### Materials and Methods

Reconstructed epidermis were topically treated with a formulation (cream) containing 1% tea tree oil concentrate or its placebo. After 24 hours of pre-incubation, epidermis were stimulated with 0.1% SDS for 30 minutes then treated again with the products for 24 hours and incubated again for 24 hours.

Culture subnatants were collected then IL-1 alpha and IL8 were measured by ELISA assay.

The results were statistically analyzed using ANOVA followed by Dunnett's test.

### Results

Tea tree oil concentrate, formulated at 1%, inhibits SDS-induced IL-1 alpha and IL-8 release.

TABLE 10
Dosage of IL-1 alpha produced by reconstructed
epidermis stimulated by PMA

| | IL-1 alpha | | IL8 | |
|---|---|---|---|---|
| | (pg/ml) | % Evolution | (pg/ml) | % Evolution |
| Placebo | 9.6 ± 0.6 | | 42.5 ± 13.7 | |
| control | | | | |
| SDS 0.1% | 84 ± 19 | +778% | 696.2 ± 241.7 | +1536% |
| THE 1% | 55.2 ± 12.2 | −34% | 478.3 ± 61.7 | −31% |

These different results demonstrate the anti-inflammatory potential of tea oil concentrate.

### Anti-Aging Activity

Skin aging is characterized by an alteration of the dermal matrix, including a reduction in the quality and quantity of matrix fibers.

The anti-aging activity of tea oil concentrate was evaluated by its ability to stimulate the expression and production of dermal matrix markers under standard conditions and under inflammatory stress conditions modeling the “inflammaging”.

### Stimulation of Gene Expression of Dermal Matrix Markers in Normal Human Fibroblasts

The effect of tea oil concentrate on the expression of dermal matrix markers was evaluated in cultured normal human dermal fibroblasts.

### Materials and Methods

Normal human dermal fibroblasts were incubated for 48 hours in the presence of 0.001% and 0.005% tea oil concentrate or 5 ng/ml TGFβ1, the positive reference of the test.

Gene expression of Collagen Ill and Fibrillin 1 was evaluated by quantitative real-time PCR. The results were statistically analyzed by one-way analysis of variance (ANOVA), followed by Dunnett's post-test.

### a. Results

Tea oil concentrate significantly stimulated the gene expression of collagen III and Fibrillin 1. These results show a densification of the dermal matrix for a skin anti-aging action.

TABLE 11
Gene expression of dermal matrix markers in fibroblasts

| | Control | Reference | |
|---|---|---|---|
| | cells | (TGFβ 1 5 ng/ml) | THE 0.005% |
| Collagen III | 0.87 ± 0.12 | 1.55 ± 0.25 | 1.47 ± 0.37 |
| (Relative Quantity | | +78% p < 0.05 | +69% p < 0.05 |
| and % induction) | | | |
| Fibrillin 1 | 0.88 ± 0.23 | 1.53 ± 0.18 | 1.94 ± 0.71 |
| (Relative Quantity | | +74% p < 0.05 | +121% p < 0.01 |
| and % induction) | | | |

### Effect in a Model of “Inflamm'Aging”

With age, low-grade, silent but chronic inflammation can develop within skin tissues. This is the consequence of exposure to external stresses accumulated over the course of a lifetime. This inflammation results in the release of inflammatory cytokines (IL1, IL6, TNF, CRP, etc.), capable of generating tissue damage and the production of reactive oxygen species, thus promoting the acceleration of the skin aging process. This phenomenon of skin aging related to an inflammatory process is called “Inflamm'aging”.

Tea oil concentrate was evaluated in an in vitro model reproducing the process of “inflammaging”: inflammation, represented by the stimulation of IL-1 alpha production, is induced on reconstructed epidermis. The conditioned media of these reconstructed epidermis are then applied to fibroblasts and the expression of dermal markers, altered by IL1a produced by the epidermis, is evaluated.

### Materials and Methods

Reconstructed epidermis were treated topically with a formulation (cream) containing 1% tea tree oil concentrate or its placebo, or systemically with 1 μM dexamethasone, an anti-inflammatory reference. After 24 hours of pre-incubation, epidermis were stimulated with 0.5 μg/ml of PMA and incubated again for 24 hours.

Culture subnatants were collected, IL-1 alpha assay was performed on the one hand, and on the other hand, these subnatants (conditioned media) were deposited on cultures of normal human dermal fibroblasts.

After 24 hours of incubation of fibroblasts in the presence of conditioned media, the expression of markers of interest was assessed by real-time quantitative PCR.

The results were statistically analyzed using ANOVA followed by Tukey's test.

### Results

Tea oil concentrate, formulated at 1%, significantly inhibited PMA-induced IL-1 alpha release in reconstructed epidermis. This effect was significantly different from placebo.

The application of conditioned media to fibroblast cultures, reconstructed epidermis stimulated by PMA, induced variations in gene expression of inflammatory markers, oxidative stress, synthesis and degradation of the dermal matrix. These results validate the model of “Inflamm'aging”.

Under these conditions, tea oil concentrate modulated the overexpression of cytokines and chemokines (CXCL1, IL8, IL6, CCL2), matrix degrading enzymes (MMP1, MMP3) and increased the gene expression level of HAS2.

These results show that the tea oil concentrate can modulate the process of “inflamm'aging”.

TABLE 12
Dosage of IL-1 alpha produced by reconstructed
epidermis stimulated by PMA

| | Interleukin-1 | | | |
|---|---|---|---|---|
| | alpha | | | |
| | (pg/mL) | % Protection | | |
| Control cells | 122 ± 4 | | | |
| Stimulated cells | 533 ± 35 | | | |
| (PMA 0.5 μg/mL) | | | | |
| Reference | 334 ± 41 | 48% | p < 0.001 | |
| (Dexamethasone 1 μM) | | | | |
| Placebo | 246 ± 15 | 70% | p < 0.001 | p < 0.05 |
| THE 1% | 105 ± 11 | 104% | p < 0.001 | |

TABLE 13
Genes modulated by the 1% Tea Oil Concentrate formula in the “Inflamm'aging”: gene
expression in fibroblasts incubated in the presence of conditioned media (Expression
level in relative quantity; % protection compared to PMA)

| | | | | Dexa. | | |
|---|---|---|---|---|---|---|
| | | Control | PMA | 1 μM | Placebo | THE 1% |
| Cytokines/ | CXCL1 | 100 | 1116 | 1503 | 381 (72%) | 96 (100%) |
| Chemokines | IL8 | 100 | 1645 | 1350 | 325 (85%) | 58 (103%) |
| | IL6 | 100 | 1817 | 305 | 509 (76%) | 179 (95%) |
| | CCL2 | 100 | 307 | 193 | 198 (53%) | 81 (109%) |
| Response to | SOD2 | 100 | 333 | 354 | 241 (39%) | 102 (99%) |
| oxidative and | | | | | | |
| cellular stress | | | | | | |
| Assembly/ | HAS2 | 100 | 78 | 104 | 100 (100%) | 150 (327%) |
| synthesis of the | | | | | | |
| extracellular | | | | | | |
| matrix | | | | | | |
| Degradation of | MMP1 | 100 | 1060 | 52 | 306 (79%) | 155 (94%) |
| the extracellular | MMP3 | 100 | 300 | 74 | 138 (81%) | 105 (98%) |
| matrix | | | | | | |

### Activity Screening on Reconstructed Skin

### Materials and Methods

A formulation (cream) containing 1% tea tree oil concentrate, or its placebo, was applied to the surface of reconstructed human skin. After 24 hours of incubation, gene expression of markers of interest for their dermal benefits was assessed by quantitative real-time PCR.

The results were statistically analyzed by Student's test t.

### Results

Tea oil concentrate, formulated at 1%, significantly increased the gene expression level of the markers MKI67 and PSMD1. These effects were significantly different from the placebo.

These two genes are involved in two distinct cellular functions: proliferation for the marker MKI67 and the proteasome for the marker PSMD1.

Aging is associated with a decrease in cell proliferative capacity. A decrease in the expression of the marker MKI67 has been observed in aged cells. By increasing the level of MKI67 gene expression, tea oil concentrate can thus promote cell proliferation, thus demonstrating anti-aging potential.

PSMD1 is a proteasome subunit that plays a key structural role by acting as a docking site for ubiquitinated substrates ready for removal. A decrease in this marker has been observed in aged fibroblasts. The decreased ability of cells to remove damaged proteins is a marker of aging. By increasing the gene expression level of PSMD1, tea oil concentrate may promote the removal of damaged proteins and thus demonstrate anti-aging potential.

TABLE 14
Genes significantly modulated by the 1% tea oil concentrate
formulation compared to placebo (set at 1.00)

| | | Relative | |
|---|---|---|---|
| Genes | Names | Quantity | P value |
| MKI67 | Antigen Ki-67 | 1.57 (+57%) | 0.0308 |
| PSMD1 | 26S proteasome non-ATPase regulatory | 1.83 (+83%) | 0.0465 |
| | subunit 1 | | |

### Pro-Barrier Potential

### Materials and Methods

A formulation (cream) containing 1% tea tree oil concentrate, or its placebo, was applied to the surface of reconstructed epidermis. After 24 hours of incubation, gene expression of markers of interest for their epidermal benefits was assessed by quantitative real-time PCR.

The results were statistically analyzed by Student's test t.

### Results

Tea oil concentrate, formulated at 1%, significantly increased the gene expression level of barrier markers (TJP1, DSC1, LOR, ZNF750, CERS3). These effects were significantly different from placebo, demonstrating a pro-barrier potential of tea oil concentrate.

TABLE 15
Genes significantly modulated by the 1% tea oil concentrate
formulation compared to placebo (set at 1.00)

| | | Relative | |
|---|---|---|---|
| Genes | Names | Quantity | P value |
| TJP1 | Tight junction ZO-1 protein | 1.70 (+70%) | 0.027 |
| DSC1 | Desmocollin-1 | 1.43 (+43%) | 0.001 |
| LOR | Loricrin | 1.47 (+47%) | 0.026 |
| ZNF750 | Zinc finger protein 750 | 1.60 (+60%) | 0.039 |
| CERS3 | Ceramide synthase 3 | 1.33 (+33%) | 0.048 |

There are different types of cell junctions within the epidermis that ensure cohesion between keratinocytes: tight junctions, desmosomes, and gap junctions. These different junctions contribute to the skin barrier function.

Tight junctions are intercellular junctions that seal keratinocytes together. The protein TJP1 is a marker of tight junctions. It is involved in controlling the paracellular diffusion of ions and solutes.

Desmosomes are localized junctions between keratinocytes in the basal, spinous, and granular layers. Desmosomes consist of three parts: an intracellular part, a transmembrane part, and an extracellular part. Desmocollin-1 is a component of desmosomes. It is a transmembrane glycoprotein belonging to the cadherin family.

Loricrin, ZNF50, and CERS3 markers are involved in the formation of the cornified envelope. During cornification, the plasma membrane of differentiating keratinocytes is replaced by the cornified envelope, which consists of insoluble structural extracellular proteins covalently bonded to each other and to lipid remodeling. This prevents excessive loss of body fluids and also provides an effective barrier against pathogens.

About 80% of the cornified envelope is made up of loricrin.

Keratinocyte differentiation is stimulated by numerous transcription factors such as ZNF50, a transcription factor known to regulate several genes encoding components of the cornified envelope and terminal differentiation of keratinocytes.

Ceramides are the predominant lipids in the skin and constitute approximately 50% of intercellular lipids by mass. They participate in the skin's barrier function and the retention of hydration. Ceramide synthases (CERS) 3 and 4 are predominant in the skin.

The tea oil concentrate, by significantly increasing the level of gene expression of these different markers, thus contributes to a pro-barrier effect.

### Activity in In-Vitro Models Related to Skin Comfort and Well-being

Psychological stress has multiple physiological and clinical impacts on the skin. The physiological impacts are varied (inflammation, impaired barrier function, impaired healing, suppressed immunity) and will lead to clinical impacts (dry/sensitive skin phenotype, redness, irritation, itching, blemishes, acne, eczema, etc.).

There are two main axes of response to stress: the sympathetic nervous system which will induce the release of neuropeptides (substance P for example) and catecholamines and the Hypothalamic-pituitary-adrenal axis (HPA axis) which will induce the release of cortisol.

Acute psychological stress can alter the permeability of the skin barrier as well as the integrity of the stratum corneum.

To evaluate the effect of tea oil concentrate on a biological comfort and well-being axis, different in vitro models of stress related to cortisol and substance P were carried out.

### Effect of Tea Oil Concentrate on Cortisol Release in a Skin Explant Model

### Materials and Methods

Skin explants were treated topically with a formulation (cream) containing 1% tea oil concentrate, or its placebo, or treated systemically (in the culture medium) with 0.001% tea oil concentrate and the positive reference metyrapone at 1 mM. The explants were incubated for 24 hours then stressed with 0.6 μM ACTH (Adreno Corticotropic Hormone), a hormone that induces cortisol production. This stress was coupled with explant culture in a 42° C. incubator to potentiate the increase in cortisol production. The explants were again treated with topical products or the active ingredient and the positive reference systemically. After 24 hours of incubation, the culture media were recovered and an ELISA assay for cortisol was performed.

The results were statistically analyzed by Student's test t.

### Results

Tea oil concentrate tested systemically in the culture medium decreases cortisol in explant culture subnatants, cortisol induced by ACTH treatment coupled with explant culture in a 42° C. incubator.

Tea tree oil concentrate, formulated at 1% versus placebo, inhibits cortisol, while the placebo increased cortisol release.

TABLE 16
Dosage of cortisol produced by skin explants stimulated
by ACTH + T° C. 42° C. (Tea oil
concentrate tested systemically in the culture medium)

| | Relative | % | |
|---|---|---|---|
| | quantification | Protection | P value |
| Control cells | 1 ± 0.17 | | |
| Stimulated cells | 1.29 ± 0.05 | | |
| (ACTH 0.6 μM + 42° C.) | | | |
| Reference | 1.08 ± 0.12 | 70% | p < 0.1 |
| (Metyrapone 1 mM) | | | |
| THE 0.001% | 1.02 ± 0.04 | 93% | p < 0.01 |

TABLE 17
Dosage of cortisol produced by skin explants stimulated
by ACTH + T° C. 42° C. (Tea oil concentrate
tested topically in formulation versus placebo)

| | | % Protection | |
|---|---|---|---|
| | Relative | (vs | P |
| | quantification | ACTH/42° C.) | value |
| Control cells | 1 ± 0.17 | | |
| Stimulated cells | 1.32 ± 0.12 | | |
| (ACTH 0.6 μM + 42° C.) | | | |
| Reference | 1.08 ± 0.12 | 73% | |
| (Metyrapone 1 mM) | | | |
| Placebo | 1.47 ± 0.27 | −50% | p < 0.1 |
| THE 1% | 1.16 ± 0.05 | 49% | |

### Effect of Tea Oil Concentrate in an Exogenous/Endogenous Stress Model Related to Skin Barrier Protection

Tea oil concentrate was evaluated in a model of keratinocytes (HaCaT) stressed with cortisone then UVA. UVA stress potentiates the transformation of cortisone (inactive molecule) into cortisol (active molecule). Cortisol is described to alter the skin barrier.

### Materials and Methods

HaCaT keratinocytes were treated with 0.0005% and 0.001% tea oil concentrate in parallel with 100 nM cortisone treatment. After 24 hours of incubation, the cells were stressed with UVA at 3 J/cm². Cell lysates were collected either immediately after irradiation for analysis of carbonylated proteins by immunostaining or 6 hours after irradiation for analysis of barrier and inflammation markers by RT-qPCR.

The results were statistically analyzed using ANOVA followed by Dunnett's test.

### Results

Cortisone stress combined with UVA increased the gene expression level of IL-1 alpha, reflecting an activation of inflammation. It also increased the amount of carbonylated proteins. It inhibited the gene expression level of barrier markers (loricrin, filaggrin and keratin 10) demonstrating the negative impact of cortisol on the skin barrier.

Tea oil concentrate showed a pro-barrier protective effect by increasing the cortisol/UVA stress-inhibited markers filaggrin, loricrin, and keratin 10. Tea oil concentrate also showed an anti-inflammatory effect on IL1a (gene expression) and decreased the amount of stress-induced protein carbonyls (damaged proteins).

This latter result can be correlated with a result observed during the activity screening on reconstructed skin where the tea oil concentrate significantly increased the level of gene expression of the marker PSMD1 (26S proteasome non-ATPase regulatory subunit 1), a proteasome marker involved in the degradation of damaged proteins.

TABLE 18
Genes significantly modulated by tea oil concentrate (RQ =
Relative Quantification - Untreated control set at 100)

| | IL-1 alpha | Loricrin | Filaggrin | Keratin 10 |
|---|---|---|---|---|
| | QR (% | QR (% | QR (% | QR (% |
| | protection) | protection) | protection) | protection) |
| Control cells | 100 ± 4 | 100 ± 10 | 100 ± 14 | 100 ± 9 |
| Stimulated cells | 377 ± 16 | 68 ± 23 | 63 ± 1 | 28 ± 9 |
| (Cortisone 100 nM + | | | | |
| UVA 3 J/cm²) | | | | |
| THE 0.0005% | 275 ± 9 | 124 ± 32 | 90 ± 7 | 44 ± 3 |
| | 37% p < 0.0001 | 174% p < 0.1 | 72% p < 0.05 | 23% p < 0.05 |

TABLE 19
Quantification of carbonylated proteins
(RQ = Relative Quantification)

| | Carbonylated | | |
|---|---|---|---|
| | proteins QR | % Protection | P value |
| Control cells | 100 ± 4 | | |
| Stimulated cells | 128 ± 10 | | |
| (Cortisone 100 nM + | | | |
| UVA 3 J/cm²) | | | |
| THE 0.0005% | 111 ± 7 | 65% | p < 0.01 |
| THE 0.001% | 100 ± 9 | 100% | P < 0.01 |

### Effect of Tea Oil Concentrate on a Cortisol Stress Model Impacting the Skin Barrier

A study was conducted on a skin explant model whose barrier was weakened by tape stripping and then stressed with cortisol. Cortisol impairs the skin barrier and delays its repair.

### Materials and Methods

The stratum corneum of skin explant was altered by tape stripping followed by 0.001% cortisol stress. After 6 hours of incubation, the explants were treated on their surface with a formulation (cream) containing 1% tea tree oil concentrate, or its placebo. 24 hours after the first application (D1), the explants were treated again with cortisol then the products 6 hours later. These treatments (cortisol+product 6 hours later) were repeated for a total of 5 treatments (DO to D4). 24 hours after the last treatment (D5), the explants were recovered in order to carry out different markings:
Hematoxylin/eosin marking for stratum corneum thickness analysis,
safranin-O-red marking for the analysis of the number of layers of the stratum corneum,
A Lucifer Yellow marking for skin barrier integrity analysis.

Results were statistically analyzed using ANOVA followed by Tukey or Dunnett test.

### Results

Alteration of the stratum corneum by tape stripping followed by repeated treatment with cortisol for several days significantly reduced the thickness of the stratum corneum and the integrity of the skin barrier.

The tea oil concentrate, tested at 1% versus placebo, shows significant efficiency on the thickness of the stratum corneum and the number of layers of the stratum corneum (histological marking) which are greater in the condition treated with the active ingredient. It also shows significant efficacy on the integrity of the skin barrier (penetration of the lucifer yellow marker).

The tea oil concentrate thus promotes the repair of the skin barrier and protects against psychological stress which alters the repair of the skin barrier.

TABLE 20
Quantification of the thickness of the stratum corneum

| | Thickness | % | | |
|---|---|---|---|---|
| | (μm) | Efficiency | P value | |
| Control explants | 13.4 ± 1.0 | | | |
| Stressed explants | 8.3 ± 0.4 | | | |
| (Tape stripping + | | | | |
| Cortisol 0.001%) | | | | |
| Placebo | 10.1 ± 1.0 | 35% | p < 0.001 | p < 0.001 |
| THE 1% | 12.8 ± 1.0 | 88% | p < 0.001 | |

TABLE 21
Quantification of the number of layers of the stratum corneum

| | Number of | % | | |
|---|---|---|---|---|
| | layers | Efficiency | P value | |
| Control explants | 9.6 ± 0.5 | | | |
| Stressed explants | 4.4 ± 0.5 | | | |
| (Tape stripping + | | | | |
| Cortisol 0.001%) | | | | |
| Placebo | 6.8 ± 0.4 | 46% | p < 0.001 | p < 0.001 |
| THE 1% | 9.1 ± 0.9 | 91% | p < 0.001 | |

TABLE 22
Analysis of skin barrier integrity via the permeability
of the fluorescent Lucifer Yellow marker

| | Fluorescence | | | |
|---|---|---|---|---|
| | intensity | % | | |
| | (RFU) | Efficiency | P value | |
| Control explants | 211 ± 31 | | | |
| Stressed explants | 509 ± 85 | | | |
| (Tape stripping + | | | | |
| Cortisol 0.001%) | | | | |
| Placebo | 401 ± 62 | 36% | p < 0.01 | p < 0.001 |
| THE 1% | 262 ± 62 | 83% | p < 0.001 | |

These different results indicate that tea oil concentrate is of great interest in the comfort of dry and sensitive skin which can be caused and amplified by psychological stress.

### Effect of Tea Oil Concentrate in a Reinnervated Epidermis Model

### Materials and Methods

iPS (induced pluripotent stem cells) cells of human origin were differentiated into sensory neurons in culture inserts. A collagen gel was then applied to the sensory neurons. After 3 weeks of culture, an explanted epidermis, dissociated from the dermis by dispase treatment, was deposited on the collagen gel. The model was incubated for 5 days, the time for reinnervation of the epidermis by sensory neurons. Then epidermis were topically treated with tea oil concentrate formulated at 0.5% and 1% in paraffin. After 24 hours of incubation, epidermis were topically treated with 10% lactic acid for 15 minutes. The culture media were then recovered and an ELISA assay for the substance P was performed.

The results were statistically analyzed using the Mann-Whitney U test.

### b. Results

Tea oil concentrate, tested at 0.5% and 1%, significantly inhibits the release of substance P, a neuropeptide involved in pain perception, induced by lactic acid in a reinnervated epidermis model.

Tea oil concentrate is therefore of interest in the comfort of sensitive skin.

TABLE 23
Dosage of substance P released by reinnervated
epidermis stressed with lactic acid

| | Substance P | | |
|---|---|---|---|
| | (pg/mL) | % Evolution | P value |
| Controlled | 629 ± 228 | | |
| epidermis | | | |
| Lactic acid stressed | 934 ± 198 | +48% | |
| epidermis 10% | | | |
| THE 0.5% | 488 ± 170 | −48% | p < 0.05 |
| THE 1% | 407 ± 178 | −56% | p < 0.05 |

By reducing the release of cortisol and substance P, and by protecting the skin from the harmful effects of cortisol, tea oil concentrate brings comfort and well-being to dry and sensitive skin.

### Example 3: In Vivo Biological Activity Tests of the Extract According to the Invention

### Concept and Protocol

### Concept

People with dry and/or sensitive skin experience symptoms at the skin (tingling, tautness, etc.) which negatively impact their skin comfort and well-being, which can lead to a psychological state of stress or a degraded emotional state and also cause a reduction in their quality of life. A vicious circle can then set in, with psychological stress itself being a source of skin problems (Farage, 2022).

The interest of a cosmetic or dermatological active ingredient in the context of this problem is to act on the symptoms related to dry and/or sensitive skin in order to limit the negative impact on skin discomfort and their quality of life on the psychological state of people and improve their emotional state and their well-being.

### Protocol

### Objective

The objective of this study is to evaluate the efficacy and emotional benefit of the extract according to the invention in comparison with a placebo in people expressing psychological discomfort/stress/bad mood due to their dry skin and/or their sensitive skin.

### Products

The extract tested is the concentrate from Example 1. The active ingredient is formulated with 1% tea oil concentrate. The details of the compositions are shown in the table below.

TABLE 24

| | Active | Placebo |
|---|---|---|
| INCI name | % Matter | % Matter |
| Aqua (Water) | qsp 100% | qsp 100% |
| Dicaprylyl carbonate & Tocopherol | 3.000 | 3.000 |
| Triisostearin | 3.000 | 3.000 |
| Sodium acrylates copolymer & Lecithin | 2.000 | 2.000 |
| Lauryl glucoside & Polyglyceryl-2 | 1.000 | 1.000 |
| dipolyhydroxystearate & Glycerin & | | |
| Aqua (water) | | |
| 1,2-hexanediol | 1.000 | 1.000 |
| Tea oil concentrate | 1.000 | — |
| Glyceryl caprylate | 0.430 | 0.430 |
| Citric acid | 0.080 | 0.080 |

### c. Parameters

Application for 28 days, 2 times a day (morning and evening), face and body (evaluations on the face).
Active comparative study (n=22, 18 dry skin/19 sensitive skin, 51.3±14.7 years [19-70]) vs Placebo (n=22, 19 dry skin/20 sensitive skin, 45.3±13.8 years [23-68]) double-blinded, randomized.
Female subject, healthy (no skin or other unstabilized pathology likely to interfere with the study, no symptoms during exploratory assessment), aged over 18 years-Phototype I to IV (Caucasian).
Subject with dry and/or sensitive skin type on the face (associated with functional and/or clinical signs) (even distribution between dry skin and sensitive skin).
Subject expressing psychological discomfort/stress/bad mood due to dry skin and/or sensitive skin.
Evaluation using a combination of clinical assessments, subjective questionnaire evaluations, and objective physiological measurements.

### Results

### Skin Sensitivity (“Sensitive Scale”)

The “Sensitive Scale” described in the literature allows the evaluation of the degree of overall skin irritation and the severity of the skin condition (Misery et al., 2014). This scale is made up of 10 items corresponding to signs felt (8 items) or apparent (2 items) related to skin discomfort: tingling, burning, warm feeling, tautness, itching, pains, general discomfort, hot flushes, redness and desquamation.

The subject can graduate the intensity of each parameter from 0 to 10.

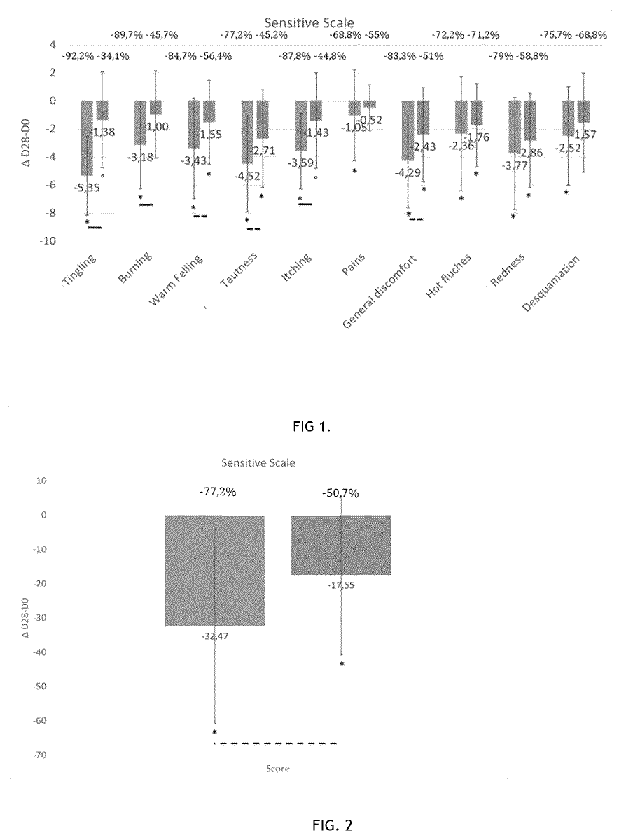

For each of the 10 parameters, the improvement is significant between T0 and T28 with the active ingredient, whereas it is only significant for 6 parameters for the placebo (see FIG. 1).

The active ingredient shows significantly improved results compared to the placebo. Indeed, the following three parameters are at the threshold of p<0.05: tingling, burning and itching.

In addition, the following three parameters as well as the overall score are at the threshold of p<0.1 (see FIG. 2): warm feeling, tautness and general discomfort.

### Prosody and Cardiac Coherence

Prosody corresponds to the emotional content of the voice and can be assessed by studying the vocal spectrum, revealing variations in several parameters. Vocal stress load was quantified using algorithmic processing.

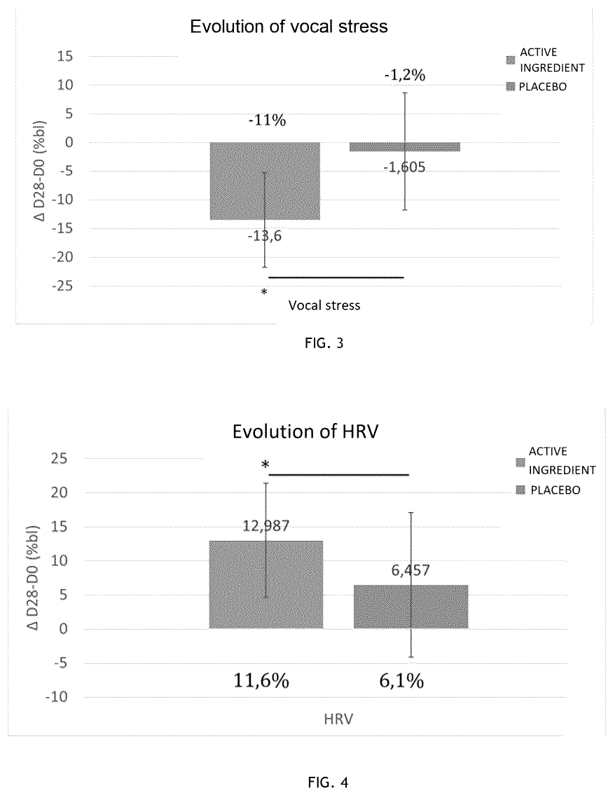

The active ingredient significantly reduces vocal stress after 28 days and the evolution compared to the placebo is significantly better (see FIG. 3).

Heart rate variability (HRV) is the cardiac measure of the interaction between the sympathetic and parasympathetic branches of the autonomic nervous system (ANS). Slight variations in HRV are commonly used in psychosociological studies of stress because they offer one of the most stable parameters of a subject's emotional tension. Stress and negative emotions are well known to disrupt cardiac coherence and induce irregularities in the HRV signal. A more positive mood should therefore increase HRV index values.

The active ingredient significantly increases HRV cardiac coherence after 28 days and the difference with the placebo is significantly better (see FIG. 4).

### Electrodermal Response

The electrodermal response (EDR) corresponds to the variation in the skin's resistance when an electrical current passes there through. It is a response that occurs following sensory or emotional arousals. This variation in the skin's electrical resistance is the result of physiological changes that may be unconscious or imperceptible to the naked eye. These neuro-vegetative changes are the consequences of the activation of the sympathetic nervous system. Indeed, following psychological (that is to say, emotional) arousal, the activity of the sweat glands increases, which in turn increases skin conductance, resulting in a very rapid EDR. This reaction occurs mainly on the plantar and palmar areas. The sweat glands are controlled by the sympathetic nervous system through the fibers of the cholinergic ganglia. In this way, the electrodermal response can be used as a physiological sign of emotional arousal.

In this study, after connecting the electrodes for the electrodermal response (EDR) on the finger, a technician asks the subject to cover their eyes with a blindfold. A patch inducing a temperature stimulus is placed on the subject's cheek before heating. After a 2-minute rest period to stabilize the parameters, the technician triggers the heating of the patch for 30 seconds to achieve the temperature stimulus and record the electrodermal response (EDR). The cheek patch is finally removed (the subject does not see the patch at any time).

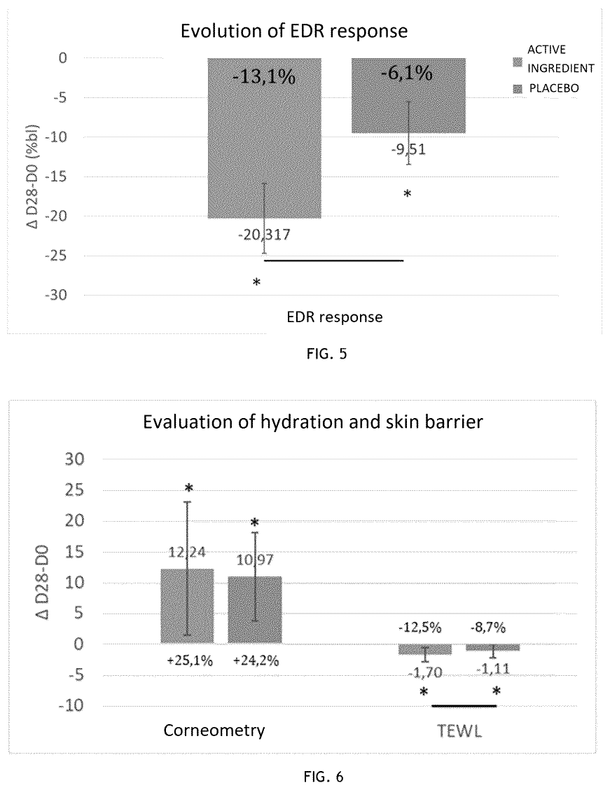

Both the active ingredient and the placebo significantly decreased the electrodermal response following a thermal stimulus after 28 days and the evolution with the active ingredient was significantly better compared to that of the placebo (see FIG. 5).

### Example 4: In Vivo Biological Activity Tests of the Extract According to the Invention

### Concept and Protocol

### 2) Concept

The purpose of this study is to evaluate the efficacy and benefit of an active ingredient on skin condition in comparison with a placebo in people with dry and/or sensitive skin conditions. The skin barrier plays a regulatory role in the skin water balance. When the skin barrier is damaged, there are disruptions in the regulation of water exchange. Water then migrates more easily to the external environment, which increases Transepidermal Water Loss (TEWL). On the other hand, if the condition of the skin barrier improves, the water loss values will decrease because the regulation of water exchange will be ensured correctly.

### Protocol

### Objective

The purpose of this study is to evaluate the efficacy and benefit of an active ingredient on skin condition in comparison with a placebo in people with dry and/or sensitive skin conditions.

### Products

The extract tested is the concentrate from Example 1. The active ingredient is formulated with 1% tea oil concentrate. The composition details are shown in the table below.

TABLE 25

| | Active | Placebo |
|---|---|---|
| INCI name | % Matter | % Matter |
| Aqua (Water) | qsp 100% | qsp 100% |
| Caprylic/capric triglyceride | 5.000 | 5.000 |
| Sclerotium gum | 1.000 | 1.000 |
| 1,2-Hexanediol | 1.000 | 1.000 |
| Tea oil concentrate | 1.000 | — |
| Caprylyl glycol | 0.300 | 0.300 |
| Citric acid | 0.004 | 0.004 |

### Parameters

Application for 28 days, 2 times a day (morning and evening), face and body (evaluations on the face).
Active ingredient comparative study (n=24, 19 visible dry skin/23 sensitive skin, 50.0±16.7 years [24-71]) vs placebo (n=24, 22 visible dry skin/22 sensitive skin, 54.2±16.8 years [24-74]), double-blinded, randomized.
Female subject, healthy (no skin or other unstabilized pathology likely to interfere with the study, no symptoms during exploratory assessment), aged over 18 years-Phototype I to IV (Caucasian).
At least 50% of subjects with visibly dry skin on the face, at least 50% of subjects with sensitive skin (assessed according to a literature questionnaire: Boyer et al., 2021) with 1 clinical sign and 1 subjective sign on at least 2 stresses.
Subjects experiencing tingling and/or warm feeling, burning and/or itching.
Subjects expressing skin discomfort ≥4 out of 10.
Evaluation: Transepidermal water loss measurements were performed using a Tewameter™ 300®. A probe consisting of two sensors is traversed by a stream of water vapor. The partial pressure difference between the two sensors is measured. This value corresponds to the evaporation rate of a volatile substance (in this case, water).

### Hydration and Barrier Function Results

Hydration is improved significantly with both the active ingredient and placebo.

The skin barrier evaluated through TEWL is improved significantly with the active ingredient and placebo (see FIG. 6). The difference active ingredient vs placebo is significant in favor of the active ingredient (Δ% (Δ Active ingredient/Δ Placebo)=−34.8%, p<0.05 (Mann-Whitney Test)).

### BIBLIOGRAPHICAL REFERENCES

Yang C., Liu X., Chen Z., Lin Y., Wang S. Comparison of Oil Content and Fatty Acid Profile of Ten New Camellia oleifera Cultivars, Journal of Lipids, 2016, 216. https://doi.org/10.1155/2016/3982486
Hu J. B., Yang G. L. Physiochemical characteristics, fatty acid profile and tocopherol composition of the oil from Camellia oleifera Abel cultivated in Henan, China, Grasas y aceites 2018, 69(2)
Wen Y., Su S.-C., Lv-yi M., Yang S.-Y., Wang Y.-W., Wang X.-N., Effects of canopy microclimate on fruit yield and quality of Camellia oleifera. Scientia Horticulturae, 2018, 235. https://doi.org/10.1016/j.scienta.2017.12.042.
Farage M. A. Psychological Aspects of Sensitive Skin: A Vicious Cycle. Cosmetics 2022, 9, 78. https://doi.org/10.3390/cosmetics9040078
Misery L., Jean-Decoster C., Mery S., Georgescu V., Sibaud V. A new ten-item questionnaire for assessing sensitive skin: the Sensitive Scale-10. Acta Derm Venereol. 2014, 94(6):635-9. doi: 10.2340/00015555-1870.
Boyer G., Belilovsky C. D., Bredif S., Baudouin C., Misery L., Bellemère G. Clinical and Instrumental Exploration of Sensitive Skin in a Pediatric Population. Cosmetics 2021, 8, 43. https://doi.org/10.3390/cosmetics8020043.

1.-12. (canceled)

13. A lipid extract of tea oil, wherein the lipid extract is a Camellia oleifera tea oil, concentrated in its unsaponifiable fraction, containing from 1.5% to 100% by weight of unsaponifiables relative to the total weight of the extract.

14. The extract according to claim 13, wherein the lipid extract has a sterol content greater than 1.5% by weight relative to the total weight of the extract.

15. The extract according to claim 13, wherein the lipid extract comprises:
β-amyrin and Δ5-avenasterol; and/or
lupeol and Δ7-stigmasterol.

16. A method for preparing the lipid extract according to claim 13, comprising the following successive steps:
a) distilling a crude or refined oil of Camellia oleifera tea with a molecular distillation device to obtain an oil concentrated in unsaponifiable fraction;
b) optionally, extracting the unsaponifiable matter from the oil concentrated in unsaponifiable fraction obtained in step a);
c) recovering the oil concentrated in unsaponifiable fraction obtained in step a) or the unsaponifiable matter obtained in step b); and
d) optionally refining the oil concentrated in unsaponifiable fraction obtained in step c) or the unsaponifiable matter obtained in step c), by deodorization and/or decolorization.

17. The method according to claim 16, wherein step b) comprises the following successive steps:
i. saponifying the concentrated tea oil into its unsaponifiable fraction obtained following step a), and
ii. extracting the unsaponifiable matter using a suitable solvent.

18. A composition comprising, as active ingredient, a lipid extract of Camellia oleifera tea oil according to claim 13 and a suitable excipient.

19. The composition according to claim 18, wherein the composition comprises from 0.01% to 20% by weight, of the lipid extract, relative to the total weight of the composition.

20. The extract according to claim 13, wherein the lipid extract contains from 1.8% to 80% by weight of unsaponifiables, relative to the total weight of the extract.

21. The extract according to claim 15, wherein the lipid extract comprises:
from 10 to 50% by weight of β-amyrin and Δ5-avenasterol, relative to the total weight of sterols; and/or
from 10 to 50% by weight of lupeol and Δ7-stigmasterol, relative to the total weight of sterols.

22. The method according to claim 17, wherein the suitable solvent of step ii. is ethyl acetate or hexane.

23. A method for preventing and/or treating disorders or pathologies of the skin and/or mucous membranes and/or appendages, comprising administering to a subject in need thereof of an effective amount of the lipid extract according to claim 13.

24. The method according to claim 23, wherein the disorders or pathologies of the skin and/or mucous membranes and/or appendages are inflammatory reactions, disorders related to intrinsic or extrinsic stress, disorders of the barrier or homeostasis, photosensitized skin, mechanical and/or thermal aggressions.

25. The method according to claim 24, wherein the intrinsic or extrinsic stress are psychological stress, stress related to a state of anxiety, stress related to radical attacks related or not to chemical or atmospheric pollution, and/or related to exposure to UV or IR.

26. The method according to claim 23, wherein the lipid extract acts as a healing agent.

27. The method according to claim 23, wherein the disorders or pathologies of the skin and/or mucous membranes and/or appendages are selected from the group consisting of dry skin, xerosis, sensitive skin and irritations.

28. The method according to claim 23, wherein the disorders or pathologies are selected from the group consisting of superficial scars, fragile lips and cheilitis, stretch marks, skin after bites, skin abrasions, spots and/or skin scabs, and fragile and sensitive skin.

29. A method for cosmetic care or cosmetic treatment for the skin to improve the condition or appearance thereof, comprising orally or topically administering to a subject in need thereof an effective amount of the lipid extract according to claim 13.

30. The method according to claim 29, wherein the method is for improving firmness, elasticity or tone of the skin; and/or for improving skin comfort.
